(19) United States
(12) Patent Application Publication
(10) Pub. No.: US 2025/0124694 A1
(43) Pub. Date: Apr. 17, 2025

(54) METHOD FOR TRAINING MODEL FOR RECOGNIZING MEDICAL IMAGE, METHOD FOR RECOGNIZING MEDICAL IMAGE, AND ELECTRONIC DEVICE

(71) Applicant: Jiangyu Kangjian Innovation Medical Technology(Chengdu) Co., Ltd., Chengdu (CN)

(72) Inventors: SHENG-CHI WENG, New Taipei (TW); Han-Wei Wu, New Taipei (TW); Chan-Pang Kuok, New Taipei (TW)

(21) Appl. No.: 18/788,126

(22) Filed: Jul. 30, 2024

(30) Foreign Application Priority Data
Oct. 17, 2023 (CN) 202311347315.9

(51) Int. Cl. G06V 10/774; A61B 3/12; A61B 3/14; G06V 10/764; G06V 10/77; G06V 20/50; G06V 20/70; G16H 50/20

(52) U.S. Cl. CPC G06V 10/7747; G06V 10/764; G06V 10/7715; G06V 20/50; G06V 20/70; G16H 50/20; A61B 3/12; A61B 3/14; G06V 2201/03

(57) ABSTRACT

The present application provides a medical image recognition method. The method includes obtaining a fundus image. A feature extraction model is invoked to extract features from the fundus image to obtain a fundus feature map. Once the fundus feature map is input into an attention mechanism model to obtain an attention-weighted feature map, a classification model is invoked to classify the attention-weighted feature map to obtain a target disease label of the fundus image. The present application also provides a method for training a medical image recognition model and an electronic device.

### TECHNICAL FIELD

The present application belongs to a field of biomedicine and relates to an image processing technology, and in particular to a method for training a model for recognizing a medical image, a method for recognizing the medical image, and an electronic device.

### BACKGROUND

In the field of medical technology, fundus images have become an important tool to assist doctors in diagnosing diseases. The fundus image includes health information about patient's eyes, including a morphology and a pathological condition of structures such as a retina, a choroid and a sclera. By observing and analyzing these fundus images, doctors can diagnose various eye diseases, such as a retinal hole, a retinal hemorrhage and a retinal detachment.

However, due to a complexity of the fundus image and a diversity of lesions, a hidden biomarker (potential lesion) in the fundus image may be vague and inconspicuous. Since it is difficult to detect the hidden biomarker from the fundus image, an accuracy and a timeliness of a diagnosis of an eye disease is affected.

### SUMMARY

In view of the above, it is necessary to provide a method for training a medical image recognition model, a method for recognizing a medical image and an electronic device.

In a first aspect, the present application provides a method for training a medical image recognition model. The method comprises obtaining a plurality of training images and a preset disease label corresponding to each training image of the plurality of training image; obtaining the medical image recognition model by training a medical image recognition network based on the plurality of training images and the preset disease label corresponding to each training image, the medical image recognition network comprising a feature extraction network, an attention mechanism module, and a classification network, and wherein training the medical image recognition network comprises: inputting each training image into the feature extraction network and obtaining a training feature map of each training image; determining a training weighted feature map of each training feature map by inputting each training feature map into the attention mechanism module; obtaining a predicted disease label corresponding to each training image by invoking the classification network to classify each training image based on the training weighted feature map of each training feature map; calculating a loss value according to the predicted disease label and the preset disease label corresponding to each training image, adjusting the feature extraction network, the attention mechanism module, and the classification network according to the loss value, and obtaining a feature extraction model corresponding to the feature extraction network, an attention mechanism model corresponding to the attention mechanism module and a classification model corresponding to the classification network; and constructing the medical image recognition model based on the feature extraction model, the attention mechanism model, and the classification model.

In some embodiments of the present application, the attention mechanism module comprises a training perception layer, and determining the training weighted feature map of each training feature map by inputting each training feature map into the attention mechanism module comprises: obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value; and generating the training weighted feature map for each training feature map based on each training feature map and the corresponding training weight vector.

In some embodiments of the present application, “obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value” comprises: performing a global average pooling processing on each training feature map and obtaining training global average pooling values; inputting each of the training global average pooling values into the training perception layer, and obtaining a training weight vector corresponding to each training feature map based on a weight of each neuron in the training perception layer for each training global average pooling value, and then obtain a training weighted feature map of each training feature map based on each training feature map and the corresponding training weight vector.

In some embodiments of the present application, the training perception layer comprises a first training perception layer and a second training perception layer, the training weight vector comprises a first training weight vector and a second training weight vector, wherein “obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value” comprises: performing a global average pooling processing on each training feature map and obtaining training global average values; inputting each of the training global average values into the first training perception layer, and obtaining a first training weight vector for each training feature map based on a weight of each neuron in the first training perception layer for each training global average value; performing a global maximum pooling processing on each training feature map and obtaining training global maximum values; inputting each of the training global maximum values into the second training perception layer, and obtaining a second training weight vector for each training feature map based on a weight of each neuron in the second training perception layer for each training global maximum value.

In some embodiments of the present application, generating the training weighted feature map for each training feature map based on each training feature map and the corresponding training weight vector comprises: obtaining a first operation feature map corresponding to each training feature map based on each training feature map and the corresponding first training weight vector; obtaining a second operation feature map corresponding to each training feature map based on each training feature map and the corresponding second training weight vector; and generating a training weighted feature map for each training feature map according to the first operation feature map and the second operation feature map corresponding to each training feature map.

Through the above implementation, the medical image recognition model is trained according to the training images (i.e., fundus images) and the preset disease labels corresponding to the training images, so that a medical image recognition network can learn an association between different disease features (for example, hidden biomarkers) in multiple fundus images and the preset disease labels, so that the trained medical image recognition model can comprehensively and accurately detect the diseases in the fundus images to be recognized, thereby effectively assisting doctors in diagnosing the disease.

In a second aspect, the present application provides a method for recognizing a medical image. The method includes obtaining a fundus image to be recognized; obtaining a target disease label of the fundus image to be recognized by invoking a medical image recognition model that has been trained to recognize the fundus image to be recognized, the medical image recognition model comprising a feature extraction model, an attention mechanism model, and a classification model, and obtaining the target disease label comprising: obtaining a fundus feature map by invoking a feature extraction model to extract features from the fundus image to be recognized; inputting the fundus feature map into the attention mechanism model, and obtaining an attention-weighted feature map; and obtaining a target disease label of the fundus image to be recognized by invoking the classification model to classify the attention-weighted feature map.

In some embodiments of the present application, the attention mechanism model comprises a perception layer, “inputting the fundus feature map into the attention mechanism model, and obtaining an attention-weighted feature map” comprises: performing a pooling processing on the fundus feature map and obtaining pooling values; inputting each of the pooling values into the perception layer, and obtaining a target weight vector of the fundus feature map based on a weight of each neuron in the perception layer for each pooling value; and generating the attention-weighted feature map based on the fundus feature map and the target weight vector.

In some embodiments of the present application, “performing a pooling processing on the fundus feature map and obtaining pooling values; inputting each of the pooling values into the perception layer, and obtaining a target weight vector of the fundus feature map based on a weight of each neuron in the perception layer for each pooling value” comprises: performing a global average pooling on the fundus feature map and obtaining global average pooling values; inputting each of the global average pooling values into the perception layer, and obtaining the target weight vector based on a weight of each neuron in the perception layer for each global average pooling value; and generating the attention-weighted feature map based on the fundus feature map and the target weight vector.

In some embodiments of the present application, the perception layer comprises a first perception layer and a second perception layer, the target weight vector comprises a first target weight vector and a second target weight vector, and “performing a pooling processing on the fundus feature map and obtaining pooling values; inputting each of the pooling values into the perception layer, and obtaining a target weight vector of the fundus feature map based on a weight of each neuron in the perception layer for each pooling value” comprises: performing a global average pooling processing on the fundus feature map and obtaining global average values; inputting each of the global average values into the first perception layer, and obtaining a first target weight vector based on a weight of each neuron in the first perception layer for each global average value; and performing a global maximum pooling processing on the fundus feature map and obtaining global maximum values; and inputting each global maximum value into the second perception layer, and obtaining a second target weight vector based on a weight of each neuron in the second perception layer for each global maximum value.

In some embodiments of the present application, generating the attention-weighted feature map based on the fundus feature map and the target weight vector comprises: determining a first attention feature map based on the fundus feature map and the first target weight vector; determining a second attention feature map based on the fundus feature map and the second target weight vector; and generating the attention-weighted feature map based on the first attention feature map and the second attention feature map.

Through the above implementation, since the medical image recognition model is trained through training images (e.g., fundus images) and preset disease labels corresponding to the training images, the medical image recognition model learns the association between different disease features (for example, hidden biomarkers) in multiple fundus images and the preset disease labels. Therefore, the medical image recognition model can comprehensively and accurately detect the diseases in the fundus images to be recognized, thereby effectively assisting doctors in diagnosing the disease.

In a third aspect, the present application provides an electronic device, comprising: at least one processor; a storage device, being stored with at least one instruction, which when executed by the at least one processor, cause the at least one processor to implement the method for training the medical image recognition model, or implement the method for recognizing the medical image.

### BRIEF DESCRIPTION OF THE DRAWINGS

FIG. 1 is a schematic diagram of a structure of an electronic device provided in one embodiment of the present application.

FIG. 2 is a flow chart of a method for recognizing a medical image provided in an embodiment of the present application.

FIG. 3 is a schematic diagram of a fundus image to be recognized provided in an embodiment of the present application.

FIG. 4 is a flow chart of generating a target weight vector provided by an embodiment of the present application.

FIG. 5 is a flow chart of generating an attention-weighted feature map provided by an embodiment of the present application.

FIG. 6 is a flow chart of a method for training a model for recognizing a medical image provided in an embodiment of the present application.

FIG. 7 is a flow chart of generating a training weight vector provided by an embodiment of the present application.

FIG. 8 is a flow chart of generating a training weighted feature map provided by an embodiment of the present application.

### DETAILED DESCRIPTION

In order to make objectives, technical solutions and advantages of the present application clearer, the present application is described in detail below with reference to the accompanying drawings and specific embodiments.

It should be noted that in the present application, “at least one” means one or more, and “a plurality of” means two or more than two. “And/or” describes a association relationship of associated objects, indicating that three relationships may exist. For example, A and/or B can mean: A exists alone, A and B exist at the same time, and B exists alone, where A and B can be singular or plural. The terms “first”, “second”, “third”, “fourth”, etc. (if any) in the specification, claims and drawings of the present application are used to distinguish similar objects, rather than to describe a specific order or a sequence.

In the embodiments of the present application, words such as “exemplary” or “for example” are used to indicate examples, illustrations or descriptions. Any embodiment or design described as “exemplary” or “for example” in the embodiments of the present application should not be interpreted as being more preferred or more advantageous than other embodiments or designs. Specifically, a use of words such as “exemplary” or “for example” is intended to present related concepts in a specific way.

In the field of medical technology, fundus images have become an important tool to assist doctors in diagnosing diseases. The fundus image includes health information about patient's eyes, including a morphology and a pathological condition of structures such as a retina, a choroid and a sclera. By observing and analyzing these fundus images, doctors can diagnose various eye diseases, such as a retinal hole, a retinal hemorrhage and a retinal detachment.

However, due to a complexity of the fundus image and a diversity of lesions, a hidden biomarker (potential lesion) in the fundus image may be blurred and inconspicuous. Since it is difficult to detect the hidden biomarker from the fundus image, an accuracy and a timeliness of a diagnosis of an eye disease is affected.

In order to solve the above technical problems, the present application provides a method for training a model for recognizing a medical image (hereinafter “medical image recognition model”), a method for recognizing the medical image, and an electronic device, which can accurately detect the hidden biomarker in the fundus images, thereby improving the accuracy and timeliness of the diagnosis of eye diseases. The method for training the model for recognizing the medical image, and the method for recognizing the medical image provided in the embodiment of the present application can be applied to one or more electronic devices.

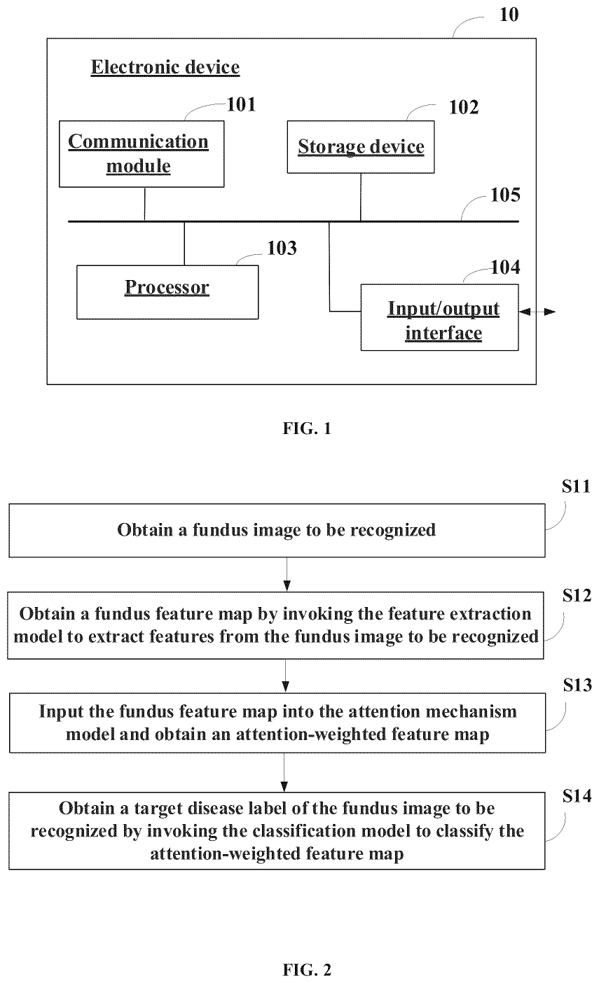

As shown in FIG. 1, it is a schematic diagram of a structure of an electronic device provided in an embodiment of the present application. An electronic device 10 can be a device such as a mobile phone, a tablet computer, a laptop computer, or a computer device. Embodiments of the present application do not limit a specific type of the electronic device.

As shown in FIG. 1, the electronic device 10 may include a communication module 101, a storage device 102, a processor 103, an input/output (I/O) interface 104, and a bus 105. The processor 103 is coupled to the communication module 101, the storage device 102, and the input/output interface 104 through the bus 105.

The communication module 101 may include a wired communication module and/or a wireless communication module. The wired communication module may provide one or more wired communication solutions such as a universal serial bus (USB), a controller area network bus (CAN). The wireless communication module may provide one or more wireless communication solutions such as a wireless fidelity (Wi-Fi), a Bluetooth (BT), a mobile communication network, a frequency modulation (FM), a near field communication (NFC) technology, an infrared (IR) technology, etc.

The storage device 102 may include one or more random access memories (RAM) and one or more non-volatile memories (NVM). The random access memory may be directly read and written by the processor 103, and may be used to store executable programs (such as machine instructions) of other running programs, and may also be used to store user data and application data. The random access memory may include a static random-access memory (SRAM), a dynamic random access memory (DRAM), a synchronous dynamic random access memory (SDRAM), a double data rate synchronous dynamic random access memory (DDR SDRAM), etc.

The non-volatile memory may also store executable programs, user data and application data, etc., and may be loaded into the random access memory in advance for direct reading and writing by the processor 110. The non-volatile memory may include a disk storage device and a flash memory.

The storage device 102 is used to store one or more computer programs. The one or more computer programs are configured to be executed by the processor 103. The one or more computer programs include a plurality of instructions, and when the plurality of instructions are executed by the processor 103, the method for training the model for recognizing the medical image and the method for recognizing the medical image executed on the electronic device 10 can be implemented.

In other embodiments, the electronic device 10 shown in FIG. 1 further includes an external storage device interface for connecting to an external storage device to expand a storage capacity of the electronic device 10.

The processor 103 may include one or more processing units, for example, the processor 103 may include an application processor (AP), a modem processor, a graphics processing unit (GPU), an image signal processor (ISP), a controller, a video codec, a digital signal processor (DSP), and/or a neural-network processing unit (NPU), etc. Different processing units may be independent devices or integrated in one or more processors.

The processor 103 provides a computing capability and a control capability. For example, the processor 103 is used to execute a computer program stored in the storage device 102 to implement the method for training the model for recognizing the medical image and the method for recognizing the medical image.

The input/output interface 104 is used to provide a channel for a user input or a user output. For example, the input/output interface 104 can be used to connect various input devices and output devices, such as a mouse, a keyboard, a touch device, a display screen, etc., so that the user can enter information or visualize information.

The bus 105 is at least used to provide a channel for a mutual communication among the communication module 101, the storage device 102, the processor 103, and the input/output interface 104 in the electronic device 10.

It is to be understood that the structure illustrated in the embodiment of the present application does not constitute a specific limitation on the electronic device 10. In other embodiments of the present application, the electronic device 10 may include more or fewer components than shown in the figure, or combine some components, or split some components, or arrange the components differently. The components shown in the figure may be implemented in hardware, software, or a combination of software and hardware.

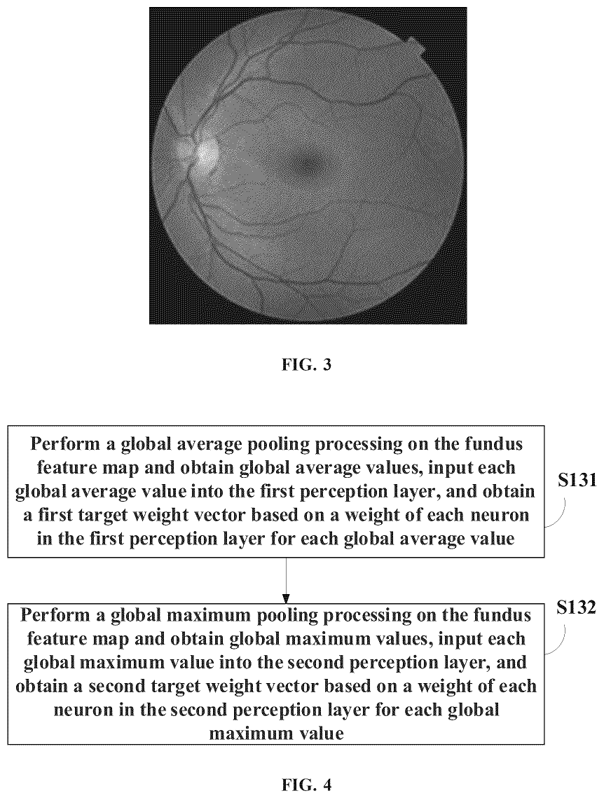

In FIG. 2, it is a flow chart of a method for recognizing a medical image provided by an embodiment of the present application. According to different requirements, an order of each block in the flow chart can be adjusted according to actual requirements, and some blocks can be omitted. The method is executed by an electronic device, such as the electronic device 1 shown in FIG. 1.

S11, the electronic device obtains a fundus image to be recognized.

In at least one embodiment of the present application, the fundus image to be recognized refers to an image that needs to be used to recognize and detect eye diseases, and the fundus image to be recognized can be obtained by photographing a fundus of an eye in front using a fundus camera. For example, as shown in FIG. 3, it is a schematic diagram of a fundus image to be recognized provided in an embodiment of the present application. As can be seen from FIG. 3, an eyeball, a retina, blood vessels, etc. can be seen from the fundus image to be recognized.

In some embodiments of the present application, after the fundus image to be recognized is obtained, the electronic device may invoke a model for recognizing a medical image (hereinafter “medical image recognition model”) that has been pre-trained to recognize the fundus image to be recognized, and obtain a target disease label of the fundus image to be recognized, among them, the medical image recognition model includes a feature extraction model, an attention mechanism model, and a classification model. The target disease label refers to a category of a disease (lesion) in the fundus image to be recognized, and there may be one or more target disease labels in the fundus image to be recognized. For example, the one or more target disease labels may refer to one or more of categories of diseases of an intraretinal fluid, a subretinal fluid, a retinal pigment cell detachment, and a macular wrinkle.

In one embodiment, obtaining the target disease label includes blocks S12-S14.

S12, the electronic device obtains a fundus feature map by invoking the feature extraction model to extract features from the fundus image to be recognized.

In at least one embodiment of the present application, the feature extraction module is a feature extraction module of a machine learning model, and the machine learning model can be a convolutional neural network (CNN) model, the CNN model may be a LeNet model, an AlexNet model, a VGG model, a GoogLeNet model, a ResNet model, a DenseNet model or other model such as an inception model, the present application does not limit on this.

In some embodiments of the present application, the machine learning model includes a convolutional network layer, a plurality of residual blocks, and a global average pooling layer. In one embodiment, the feature extraction module may include the convolutional network layer, the plurality of residual blocks, and the global average pooling layer. The convolutional network layer includes a series of convolution kernels (filters), the plurality of residual blocks are repeatedly stacked, each residual block includes batch normalization (BN) layers, activation functions (ReLu), and a plurality of convolutional layers, and each convolutional layer in each residual block is connected to one batch normalization layer and one activation function.

In this embodiment, the electronic device obtaining the fundus feature map by invoking the feature extraction model to extract features from the fundus image to be recognized, includes: invoking the convolutional network layer to perform a first feature extraction on the fundus image to be recognized, and obtaining a first feature map; performing a feature fusion operation on the first feature map through the plurality of residual blocks, and obtaining a target fused feature map; inputting the target fused feature map into the global average pooling layer and obtaining the fundus feature map. Among them, a process of performing the feature fusion operation on the first feature map through the plurality of residual blocks, and obtaining the target fused feature map includes: invoking one of the plurality of residual blocks to perform a second feature extraction on the first feature map, and obtaining a second feature map, performing a feature fusion on the first feature map and the second feature map, and obtaining a first fused feature map; invoking another residual block of the plurality of residual blocks to perform a third feature extraction on the first fused feature map, and obtaining a third feature map, performing a feature fusion on the first fused feature map and the third feature map, and obtaining a second fused feature map, repeating the above blocks until a feature map output by a last residual block of the plurality of residual blocks is obtained, and determining a feature map obtained by performing a feature fusion on a feature map output by the last residual block and a fused feature map input into the last residual block as the target fused feature map.

An operation of invoking the residual blocks for performing the feature extraction can refer to a relevant technology. The feature fusion can be performed by adding or subtracting two feature maps or concatenating (concatenate or concat) according to channel dimensions. The present application does not limit the method of feature fusion.

S13, the electronic device inputs the fundus feature map into the attention mechanism model and obtains an attention-weighted feature map.

In at least one embodiment of the present application, the attention mechanism model includes a pooling layer, and the pooling layer can be one or more of following pooling layers: a global average pooling (GAP) layer, a global maximum pooling (GMP) layer.

In some embodiments of the present application, the attention mechanism model includes a pooling layer and a perception layer. In one embodiment, the electronic device inputs the fundus feature map into the attention mechanism model and obtains the attention-weighted feature map by: performing a pooling processing on the fundus feature map and obtaining pooling values; inputting each pooling value into the perception layer, and obtaining a target weight vector of the fundus feature map based on a weight of each neuron in the perception layer for each pooling value; and generating the attention-weighted feature map based on the fundus feature map and the target weight vector.

Specifically, if the pooling layer in the attention mechanism model is the global average pooling layer, the pooling values are global average pooling values, the electronic device performs a global average pooling on the fundus feature map and obtains the global average pooling values; inputs each global average pooling value into the perception layer, and obtains the target weight vector based on the weight of each neuron in the perception layer for each global average pooling value; and generates the attention-weighted feature map based on the fundus feature map and the target weight vector.

Among them, the perception layer can be a fully connected layer, and a method for generating the attention-weighted feature map based on the fundus feature map and the target weight vector can be set according to actual needs, and the present application does not limit this. For example, the fundus feature map and the target weight vector can be multiplied, added, or subtracted to obtain the attention-weighted feature map.

In one embodiment, important features (such as hidden biomarkers) and background information in the fundus feature map are recognized and distinguished, and higher weights are assigned to the important features in the fundus feature map, so that a size of elements in the target weight vector can reflect an importance of each feature in the fundus feature map. By generating the attention-weighted feature map based on the fundus feature map and the target weight vector, it makes the important features in the attention-weighted feature map more obvious than unimportant features such as the background information.

S14, the electronic device obtains a target disease label of the fundus image to be recognized by invoking the classification model to classify the attention-weighted feature map.

In at least one embodiment machine learning model of the present application, the classification model is a classification module of a machine learning model, and the machine learning model can be a convolutional neural network (CNN) model, the CNN model may be a LeNet model, an AlexNet model, a VGG model, a GoogLeNet model, a ResNet model, a DenseNet model or other model such as an inception model, the present application does not limit on this.

In some embodiments of the present application, the classification model may include a fully connection (FC) layer and an activation function. The activation function may be a sigmoid function or a softmax function, which is not limited in the present application.

In other embodiments of the present application, the classification model may include a fully connected layer and a dense layer.

In some embodiments of the present application, the classification model classifies the attention-weighted feature map and obtains a plurality of probability values, each probability value corresponds to a preset disease label. The classification model determines a target probability value from the plurality of probability values, and determines the preset disease label corresponding to the target probability value as the target disease label.

Among them, the preset disease label can be one or more of categories of diseases of an intraretinal fluid, a subretinal fluid, a retinal pigment cell detachment, and a macular wrinkle. The method of determining the target probability value from the plurality of probability values can be set according to actual needs, and the present application does not limit this. For example, the plurality of probability values output by the classification model includes a probability value 0.1 of the intraretinal fluid, a probability value 0.2 of the subretinal fluid, a probability value 0.01 of the retinal pigment cell detachment, and a probability value 0.9 of the macular wrinkle. The maximum probability value 0.9 can be used as the target probability value, and the macular wrinkle can be used as the target disease label.

In this embodiment, since important features in the attention-weighted feature map are more obvious than unimportant features such as background information, classifying the attention-weighted feature map through the classification model can improve an accuracy of the target disease label.

Through the above implementation, since the medical image recognition model is trained using training images (i.e., fundus images) and preset disease labels corresponding to the training images, the medical image recognition model learns an association between different disease features (for example, hidden biomarkers) in a plurality of fundus images and preset disease labels. Therefore, the medical image recognition model can comprehensively and accurately detect the diseases in the fundus images to be recognized, thereby effectively assisting doctors in diagnosing the disease.

In at least one embodiment of the present application, if the pooling layer of the attention mechanism model includes the global average pooling layer and the global maximum pooling layer, the perception layer includes a first perception layer and a second perception layer, and the pooling values includes a global average value and a global maximum value, as shown in FIG. 4, it is a flowchart of generating a target weight vector provided by an embodiment of the present application, which specifically includes the following blocks:

S131, the electronic device performs a global average pooling processing on the fundus feature map and obtains global average values, inputs each global average value into the first perception layer, and obtains a first target weight vector based on a weight of each neuron in the first perception layer for each global average value.

In some embodiments of the present application, the electronic device performs the global average pooling processing on the fundus feature map and obtains the first target weight vector by: calculating a global average value of all pixel values of each channel in the fundus feature map, and obtaining a plurality of global average values of all channels in the fundus feature map; inputting the plurality of global average values to the first perception layer, and obtaining the first target vector based on the weight of each neuron in the first perception layer for each global average. For example, if a size of the fundus feature map is H*W*C, where H represents a height of the fundus feature map, W represents a width of the fundus feature map, and C represents a number of channels in the fundus feature map. The electronic device calculates the global average value of all pixel values of each channel in the fundus feature map and obtains C global average values, and inputs the C global average values to the first perception layer, and obtains the first target weight vector of a size 1*C based on the weight of each neuron in the first perception layer for each global average.

S132, the electronic device performs a global maximum pooling processing on the fundus feature map and obtains global maximum values, inputs each global maximum value into the second perception layer, and obtains a second target weight vector based on a weight of each neuron in the second perception layer for each global maximum value.

In some embodiments of the present application, the electronic device performs the global maximum pooling processing on the fundus feature map and obtains the second target weight vectors by: determining the global maximum value of all pixel values of each channel in the fundus feature map, and obtaining a plurality of global maximum values of all channels in the fundus feature map; inputting the plurality of global maximum values to the second perception layer, and obtaining the second target vector based on a weight of each neuron in the second perception layer for each global maximum value. For example, following the above embodiment, if the size of the fundus feature map is H*W*C, where H represents the height of the fundus feature map, W represents the width of the fundus feature map, and C represents the number of channels of the fundus feature map. The electronic device traverses all pixel values of each channel in the fundus feature map, and determines the global maximum value of all pixel values of each channel and obtain C global maximum values, and inputs the C global maximum values to the second perception layer, and obtains the second target weight vector of a size 1*C based on the weight of each neuron in the second perception layer for each global maximum value.

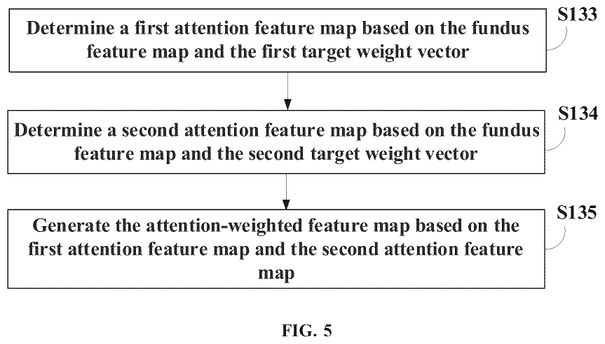

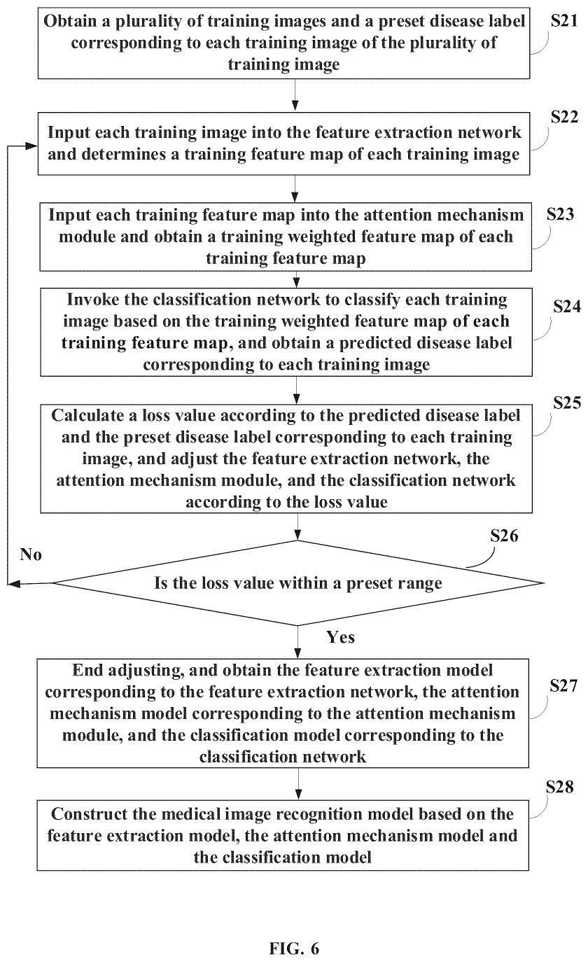

In at least one embodiment of the present application, as shown in FIG. 5, a flowchart of generating an attention-weighted feature map provided by an embodiment of the present application is shown, which specifically includes the following blocks.

S133, the electronic device determines a first attention feature map based on the fundus feature map and the first target weight vector.

In some embodiments of the present application, since the first target weight vector includes the global average value (weight) corresponding to each channel in the fundus feature map, the electronic device can determine the first channel feature map corresponding to each channel by performing an operation such as a multiplication or an addition on the pixel values of pixel points of each channel in the fundus feature map with the corresponding global average value in the first target weight vector, and obtain a plurality of first channel feature maps; determining the first attention feature map by adding the pixel values of the corresponding pixel points in the plurality of first channel feature maps.

S134, the electronic device determines a second attention feature map based on the fundus feature map and the second target weight vector.

In some embodiments of the present application, since the second target weight vector includes the global maximum value corresponding to each channel in the fundus feature map, the electronic device can determine a second channel feature map corresponding to each channel by performing an operation such as a multiplication or an addition on the pixel values of the pixel points of each channel in the fundus feature map with the corresponding global maximum value in the second target weight vector, and obtain a plurality of second channel feature maps; and obtain the second attention feature map by adding the pixel values of the corresponding pixel points in the plurality of second channel feature maps.

S135, the electronic device generates the attention-weighted feature map based on the first attention feature map and the second attention feature map.

In some embodiments of the present application, the electronic device fuses the first attention feature map and the second attention feature map to obtain the attention-weighted feature map. The first attention feature map and the second attention feature map may be fused by concatenating or concating according to channel dimensions to obtain the attention-weighted feature map.

For example, a process of obtaining the first attention feature map or the second attention feature map can refer to a formula (1):

M_{c}(x,y)=Σₖw^{c}ₖfₖ(x,y); (1)

For the first attention feature map, M_{c}(x, y) represents a pixel value at a position (x, y) in the first attention feature map, w^{c}ₖ represents the global average value corresponding to the kth channel in the first target weight vector, fₖ(x, y) represents a pixel value at a position (x, y) in the first channel feature map of the kth channel.

For the second attention feature map, M_{c}(x, y) represents a pixel value at a position (x, y) in the second attention feature map, w^{c}ₖ represents the global maximum value corresponding to the kth channel in the second target weight vector, fₖ(x, y) represents a pixel value at a position (x, y) in the second channel feature map of the kth channel.

In some embodiments of the present application, before using the medical image recognition model that has been trained to recognize the fundus image to be recognized, it is necessary to train a network for recognizing the medical image (hereinafter “medical image recognition network”) which is corresponding to the medical image recognition model, among them, the medical image recognition network includes a network for a feature extraction (hereinafter “feature extraction network”) corresponding to the feature extraction model, an attention mechanism module corresponding to the attention mechanism model, and a classification network corresponding to the classification model. As shown in FIG. 6, it is a flow chart of a method for training a medical image recognition model provided by an embodiment of the present application. According to different requirements, an order of each block in the flow chart can be adjusted according to actual detection requirements, and some blocks can be omitted. The method can be executed by an electronic device, such as the electronic device 1 shown in FIG. 1.

S21, the electronic device obtains a plurality of training images and a preset disease label corresponding to each training image of the plurality of training image.

In some embodiments of the present application, each training image is a fundus image. An introduction of the preset disease label can refer to block S14, and each training image can have a plurality of preset disease labels. In some embodiments of the present application, in order to ensure the accuracy of the preset disease label, each preset disease label can be determined by a doctor through a diagnosis of a fundus image and an optical coherence tomography (OCT) image of a patient.

In some embodiments of the present application, the preset disease label can also be obtained by detecting each training image through a convolutional neural network model. The convolutional neural network model is pre-trained through optical synchronous tomography images and disease labels obtained by doctors from each optical synchronous tomography image diagnosis. The pre-trained convolutional neural network model can accurately detect each training image, thereby improving a convenience and an accuracy of obtaining the preset disease label.

In one embodiment, the training of the medical image recognition network based on the plurality of training images and the preset disease label corresponding to each training image may include blocks S22-S28.

S22, the electronic device inputs each training image into the feature extraction network and determines a feature map (hereinafter “training feature map”) of each training image, such that a plurality training feature maps are obtained.

In at least one embodiment of the present application, the feature extraction model is a classification module of a machine learning model. If the machine learning model is a convolutional neural network (CNN) model, the CNN model may be a LeNet model, an AlexNet model, a VGG model, a GoogLeNet model, a ResNet model, a DenseNet model or other model such as an inception model, the present application does not limit on this.

In some embodiments of the present application, a process of generating each training feature map is substantially the same as the process of generating the fundus feature map, and thus the present application will not repeat the description.

S23, the electronic device inputs each training feature map of the plurality of training feature maps into the attention mechanism module and obtains a weighted feature map (hereinafter “training weighted feature map”) of each training feature map, such that a plurality of training weighted feature maps are obtained.

The attention mechanism module includes a pooling layer, which can be one or more of the following pooling layers: a global average pooling (GAP) layer, a global maximum pooling (GMP) layer.

In some embodiments of the present application, the attention mechanism module includes a pooling layer (hereinafter “training pooling layer”) and a perception layer (hereinafter “training perception layer”), among them, the electronic device inputs each training feature map into the attention mechanism module and obtains a training weighted feature map of each training feature map, by: obtaining a plurality of pooling values (hereinafter “training pooling values”) by performing a pooling processing on each training feature map, inputting each training pooling value of the plurality of training pooling values into the training perception layer, and obtaining a weight vector (hereinafter “training weight vector”) corresponding to each training feature map based on the weight of each neuron in the training perception layer for each training pooling value, and generating the training weighted feature map for each training feature map based on each training feature map and the corresponding training weight vector.

Specifically, if the training pooling layer in the attention mechanism module is the global average pooling layer (hereinafter “training global average pooling layer”), the training pooling value is a global average pooling value (hereinafter “training global average pooling value”), the electronic device performs a global average pooling processing on each training feature map and obtains training global average pooling values, and inputs each training global average pooling value into the training perception layer, and obtains a training weight vector corresponding to each training feature map based on the weight of each neuron in the training perception layer for each training global average pooling value, and then obtain a training weighted feature map of each training feature map based on each training feature map and the corresponding training weight vector.

In this embodiment, a method of obtaining the training weighted feature map of each training feature map based on each training feature map and the corresponding training weight vector can be set according to actual needs, and the present application does not limit this. For example, each training feature map can be multiplied by the corresponding training weight vector to obtain the training weighted feature map of each training feature map.

S24, the electronic device invokes the classification network to classify each training image based on the training weighted feature map of each training feature map, and obtains a predicted disease label corresponding to each training image, such that a plurality of predicted disease labels corresponding to the plurality of training images are obtained.

In some embodiments of the present application, a process of obtaining the predicted disease labels is substantially the same as the process of obtaining the target disease labels, and thus the present application will not repeat the description.

S25, the electronic device calculates a loss value according to the predicted disease label and the preset disease label corresponding to each training image, and adjusts the feature extraction network, the attention mechanism module, and the classification network according to the loss value.

In some embodiments of the present application, the loss value may be a cross entropy loss value. The method of calculating the cross entropy loss value may refer to a calculation formula of a cross entropy loss function in the related art, and the present application will not describe it in detail here.

In some embodiments of the present application, parameters such as a learning rate, a weight, and a bias in the feature extraction network can be adjusted, parameters such as a pooling window and a block size in the attention mechanism module can be adjusted, and parameters such as a weight and a bias of a fully connected layer in the classification network can be adjusted.

S26, the electronic device determines whether the loss value is within a preset range.

In this embodiment, the preset range can be set according to actual needs, and the present application does not limit this. For example, the preset range can be [0.1, 0.3]. If the loss value is not within the preset range, the process returns to block S22. If the loss value is within the preset range, block S27 is executed.

S27, the electronic device ends adjusting, and obtains the feature extraction model corresponding to the feature extraction network, the attention mechanism model corresponding to the attention mechanism module, and the classification model corresponding to the classification network.

S28, the electronic device constructs the medical image recognition model based on the feature extraction model, the attention mechanism model and the classification model.

In this embodiment, the electronic device can combine or splice the feature extraction model, the attention mechanism model and the classification model to obtain the medical image recognition model.

Through the above implementation, the medical image recognition model is trained according to the training images (i.e., fundus images) and the preset disease labels corresponding to the training images, so that the medical image recognition network can learn the association between different disease features (for example, hidden biomarkers) in multiple fundus images and the preset disease labels, so that the trained medical image recognition model can comprehensively and accurately detect the diseases in the fundus images to be recognized, thereby effectively assisting doctors in diagnosing the disease.

In at least one embodiment of the present application, if the pooling layer in the attention mechanism model includes a global average pooling layer and a global maximum pooling layer, the training perception layer includes a first training perception layer and a second training perception layer, and the training pooling value includes a global average value (hereinafter “training global average value”) and a global maximum value (hereinafter “training global maximum value”), as shown in FIG. 7, it is a flowchart for generating a training weight vector provided by an embodiment of the present application, which specifically includes the following blocks:

S231, the electronic device performs a global average pooling processing on each training feature map and obtains training global average values, inputs each training global average value into the first training perception layer, and obtains a first training weight vector for each training feature map based on the weight of each neuron in the first training perception layer for each training global average value.

In some embodiments of the present application, a process of obtaining the first training weight vector is substantially the same as the process of obtaining the first target weight vector, and thus the description thereof will not be repeated in the present application.

S232, the electronic device performs a global maximum pooling processing on each training feature map and obtains training global maximum values, inputs each training global maximum values into the second training perception layer, and obtains a second training weight vector for each training feature map based on the weight of each neuron in the second training perception layer for each training global maximum value.

In some embodiments of the present application, a process of obtaining the second training weight vector is substantially the same as the process of obtaining the second target weight vector, and thus the description thereof will not be repeated in the present application.

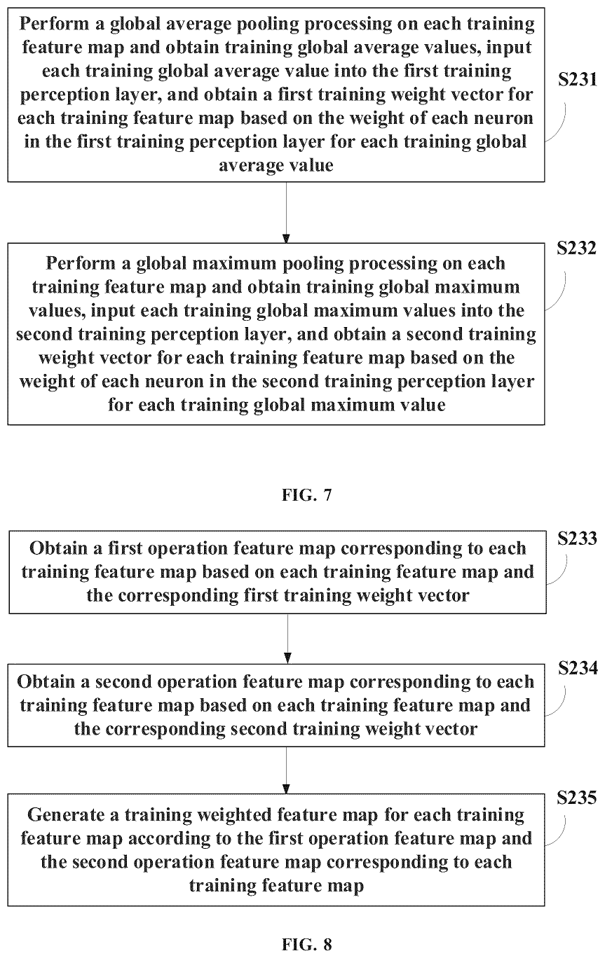

In at least one embodiment of the present application, as shown in FIG. 8, it is a flow chart of generating a training weighted feature map provided by an embodiment of the present application, which specifically includes the following blocks:

S233, the electronic device obtains a first feature map (hereinafter “first operation feature map”) corresponding to each training feature map based on each training feature map and the corresponding first training weight vector.

In some embodiments of the present application, a process of obtaining the first operation feature map is basically the same as the process of obtaining the first attention feature map, so the present application will not repeat the description.

S234, the electronic device obtains a second feature map (hereinafter “second operation feature map”) corresponding to each training feature map based on each training feature map and the corresponding second training weight vector.

In some embodiments of the present application, a process of obtaining the second operation feature map is basically the same as the process of obtaining the second attention feature map, so the present application will not repeat the description.

S235, the electronic device generates a weighted feature map (hereinafter “training weighted feature map”) for each training feature map according to the first operation feature map and the second operation feature map corresponding to each training feature map.

In some embodiments of the present application, the electronic device fuses each first operation feature map and the corresponding second operation feature map to obtain the training weighted feature map of each training feature map. Among them, each first operation feature map and the corresponding second operation feature map can be fused by concatenating or concating according to channel dimensions to obtain the training weighted feature map of each training feature map.

An embodiment of the present application further provides a computer-readable storage medium, on which a computer program is stored. The computer program includes program instructions. The method implemented when the program instructions are executed can refer to the methods in the above-mentioned embodiments of the present application.

The computer-readable storage medium may be an internal storage device of the electronic device described in the above embodiment, such as a hard disk or memory of the electronic device. The computer-readable storage medium may also be an external storage device of the electronic device, such as a plug-in hard disk, a smart media card (SMC), a secure digital (SD) card, a flash card, etc., provided on the electronic device.

In some embodiments, the computer-readable storage medium may include a program storage area and a data storage area, among them, the program storage area may store an operating system, an application required for at least one function, etc.; the data storage area may store data created according to an use of the electronic device, etc.

In the above embodiments, the description of each embodiment has its own emphasis. For parts that are not described or recorded in detail in a certain embodiment, reference can be made to the relevant descriptions of other embodiments.

Those of ordinary skill in the art can understand that units and algorithm blocks of each example described in conjunction with the embodiments disclosed herein can be implemented in electronic hardware, or a combination of computer software and electronic hardware. Whether these functions are performed in hardware or software depends on the specific application and design constraints of the technical solution. Professional and technical personnel can use different methods to implement the described functions for each specific application, but such implementation should not be considered to be beyond the scope of the present application.

The above embodiments are only used to illustrate the technical solutions of the present application, rather than to limit them. Although the present application has been described in detail with reference to the aforementioned embodiments, those skilled in the art should understand that they can still modify the technical solutions described in the aforementioned embodiments, or make equivalent replacements for some of the technical features therein. These modifications or replacements do not deviate the essence of the corresponding technical solutions from the spirit and scope of the technical solutions of the embodiments of the present application, and should all be included in the protection scope of the present application.

What is claimed is:

1. A method for training a medical image recognition model, comprising:
obtaining a plurality of training images and a preset disease label corresponding to each training image of the plurality of training image;
obtaining the medical image recognition model by training a medical image recognition network based on the plurality of training images and the preset disease label corresponding to each training image, the medical image recognition network comprising a feature extraction network, an attention mechanism module, and a classification network, and training the medical image recognition network comprising:
inputting each training image into the feature extraction network and obtaining a training feature map of each training image;
determining a training weighted feature map of each training feature map by inputting each training feature map into the attention mechanism module;
obtaining a predicted disease label corresponding to each training image by invoking the classification network to classify each training image based on the training weighted feature map of each training feature map;
calculating a loss value according to the predicted disease label and the preset disease label corresponding to each training image, adjusting the feature extraction network, the attention mechanism module, and the classification network according to the loss value, and obtaining a feature extraction model corresponding to the feature extraction network, an attention mechanism model corresponding to the attention mechanism module and a classification model corresponding to the classification network; and
constructing the medical image recognition model based on the feature extraction model, the attention mechanism model, and the classification model.

2. The medical image recognition model training method according to claim 1, wherein the attention mechanism module comprises a training perception layer, and determining the training weighted feature map of each training feature map by inputting each training feature map into the attention mechanism module comprises:
obtaining a plurality of training pooling values by performing a pooling processing on each training feature map;
inputting each training pooling value of the plurality of training pooling values into the training perception layer, and obtaining a training weight vector corresponding to each training feature map based on a weight of each neuron in the training perception layer for each training pooling value; and
generating the training weighted feature map for each training feature map based on each training feature map and the corresponding training weight vector.

3. The medical image recognition model training method according to claim 2, wherein “obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value” comprises:
performing a global average pooling processing on each training feature map and obtaining training global average pooling values;
inputting each of the training global average pooling values into the training perception layer, and obtaining a training weight vector corresponding to each training feature map based on a weight of each neuron in the training perception layer for each training global average pooling value, and then obtain a training weighted feature map of each training feature map based on each training feature map and the corresponding training weight vector.

4. The medical image recognition model training method according to claim 2, wherein the training perception layer comprises a first training perception layer and a second training perception layer, the training weight vector comprises a first training weight vector and a second training weight vector, wherein “obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value” comprises:
performing a global average pooling processing on each training feature map and obtaining training global average values;
inputting each of the training global average values into the first training perception layer, and obtaining a first training weight vector for each training feature map based on a weight of each neuron in the first training perception layer for each training global average value;
performing a global maximum pooling processing on each training feature map and obtaining training global maximum values;
inputting each of the training global maximum values into the second training perception layer, and obtaining a second training weight vector for each training feature map based on a weight of each neuron in the second training perception layer for each training global maximum value.

5. The medical image recognition model training method according to claim 4, wherein generating the training weighted feature map for each training feature map based on each training feature map and the corresponding training weight vector comprises:
obtaining a first operation feature map corresponding to each training feature map based on each training feature map and the corresponding first training weight vector;
obtaining a second operation feature map corresponding to each training feature map based on each training feature map and the corresponding second training weight vector; and
generating a training weighted feature map for each training feature map according to the first operation feature map and the second operation feature map corresponding to each training feature map.

6. A method for recognizing a medical image, comprising:
obtaining a fundus image to be recognized;
obtaining a target disease label of the fundus image to be recognized by invoking a medical image recognition model that has been trained to recognize the fundus image to be recognized, the medical image recognition model comprising a feature extraction model, an attention mechanism model, and a classification model, and obtaining the target disease label comprising:
obtaining a fundus feature map by invoking a feature extraction model to extract features from the fundus image to be recognized;
inputting the fundus feature map into the attention mechanism model, and obtaining an attention-weighted feature map; and
obtaining a target disease label of the fundus image to be recognized by invoking the classification model to classify the attention-weighted feature map.

7. The medical image recognition method according to claim 6, wherein the attention mechanism model comprises a perception layer, “inputting the fundus feature map into the attention mechanism model, and obtaining an attention-weighted feature map” comprises:
performing a pooling processing on the fundus feature map and obtaining pooling values;
inputting each of the pooling values into the perception layer, and obtaining a target weight vector of the fundus feature map based on a weight of each neuron in the perception layer for each pooling value; and
generating the attention-weighted feature map based on the fundus feature map and the target weight vector.

8. The medical image recognition method according to claim 7, wherein “performing a pooling processing on the fundus feature map and obtaining pooling values; inputting each of the pooling values into the perception layer, and obtaining a target weight vector of the fundus feature map based on a weight of each neuron in the perception layer for each pooling value” comprises:
performing a global average pooling on the fundus feature map and obtaining global average pooling values;
inputting each of the global average pooling values into the perception layer, and obtaining the target weight vector based on a weight of each neuron in the perception layer for each global average pooling value; and
generating the attention-weighted feature map based on the fundus feature map and the target weight vector.

9. The medical image recognition method according to claim 7, wherein the perception layer comprises a first perception layer and a second perception layer, the target weight vector comprises a first target weight vector and a second target weight vector, and “performing a pooling processing on the fundus feature map and obtaining pooling values; inputting each of the pooling values into the perception layer, and obtaining a target weight vector of the fundus feature map based on a weight of each neuron in the perception layer for each pooling value” comprises:
performing a global average pooling processing on the fundus feature map and obtaining global average values;
inputting each of the global average values into the first perception layer, and obtaining a first target weight vector based on a weight of each neuron in the first perception layer for each global average value; and
performing a global maximum pooling processing on the fundus feature map and obtaining global maximum values; and
inputting each global maximum value into the second perception layer, and obtaining a second target weight vector based on a weight of each neuron in the second perception layer for each global maximum value.

10. The medical image recognition method according to claim 9, wherein generating the attention-weighted feature map based on the fundus feature map and the target weight vector comprises:
determining a first attention feature map based on the fundus feature map and the first target weight vector;
determining a second attention feature map based on the fundus feature map and the second target weight vector; and
generating the attention-weighted feature map based on the first attention feature map and the second attention feature map.

11. An electronic device, comprising:
at least one processor;
a storage device stored with at least one instruction, which when executed by the at least one processor, cause the at least one processor to:
obtain a plurality of training images and a preset disease label corresponding to each training image of the plurality of training image;
obtain the medical image recognition model by training a medical image recognition network based on the plurality of training images and the preset disease label corresponding to each training image, the medical image recognition network comprising a feature extraction network, an attention mechanism module, and a classification network, and wherein the at least one processor trains the medical image recognition network by:
inputting each training image into the feature extraction network and obtaining a training feature map of each training image;
determining a training weighted feature map of each training feature map by inputting each training feature map into the attention mechanism module;
obtaining a predicted disease label corresponding to each training image by invoking the classification network to classify each training image based on the training weighted feature map of each training feature map;
calculating a loss value according to the predicted disease label and the preset disease label corresponding to each training image, adjusting the feature extraction network, the attention mechanism module, and the classification network according to the loss value, and obtaining a feature extraction model corresponding to the feature extraction network, an attention mechanism model corresponding to the attention mechanism module and a classification model corresponding to the classification network; and
constructing the medical image recognition model based on the feature extraction model, the attention mechanism model, and the classification model.

12. The electronic device according to claim 11, wherein the attention mechanism module comprises a training perception layer, and the at least one processor determines the training weighted feature map of each training feature map by inputting each training feature map into the attention mechanism module by:
obtaining a plurality of training pooling values by performing a pooling processing on each training feature map;
inputting each training pooling value of the plurality of training pooling values into the training perception layer, and obtaining a training weight vector corresponding to each training feature map based on a weight of each neuron in the training perception layer for each training pooling value; and
generating the training weighted feature map for each training feature map based on each training feature map and the corresponding training weight vector.

13. The electronic device according to claim 12, wherein “obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value” comprises:
performing a global average pooling processing on each training feature map and obtaining training global average pooling values;
inputting each of the training global average pooling values into the training perception layer, and obtaining a training weight vector corresponding to each training feature map based on a weight of each neuron in the training perception layer for each training global average pooling value, and then obtain a training weighted feature map of each training feature map based on each training feature map and the corresponding training weight vector.

14. The electronic device according to claim 12, wherein the training perception layer comprises a first training perception layer and a second training perception layer, the training weight vector comprises a first training weight vector and a second training weight vector, wherein “obtaining a plurality of training pooling values by performing a pooling processing on each training feature map; inputting each training pooling value of the plurality of training pooling values into the training perception layer, and obtaining a training weight vector corresponding to each training feature map based on a weight of each neuron in the training perception layer for each training pooling value” comprises:
performing a global average pooling processing on each training feature map and obtaining training global average values;
inputting each of the training global average values into the first training perception layer, and obtaining a first training weight vector for each training feature map based on a weight of each neuron in the first training perception layer for each training global average value;
performing a global maximum pooling processing on each training feature map and obtaining training global maximum values;
inputting each of the training global maximum values into the second training perception layer, and obtaining a second training weight vector for each training feature map based on a weight of each neuron in the second training perception layer for each training global maximum value.

15. The electronic device according to claim 14, wherein the at least one processor generates the training weighted feature map for each training feature map based on each training feature map and the corresponding training weight vector by:
obtaining a first operation feature map corresponding to each training feature map based on each training feature map and the corresponding first training weight vector;
obtaining a second operation feature map corresponding to each training feature map based on each training feature map and the corresponding second training weight vector; and
generating a training weighted feature map for each training feature map according to the first operation feature map and the second operation feature map corresponding to each training feature map.

16. The electronic device according claim 11, wherein the at least one processor is further caused to:
obtain a fundus image to be recognized;
obtain a target disease label of the fundus image to be recognized by invoking a medical image recognition model that has been trained to recognize the fundus image to be recognized, the medical image recognition model comprising a feature extraction model, an attention mechanism model, and a classification model, and obtaining the target disease label comprising:
obtaining a fundus feature map by invoking a feature extraction model to extract features from the fundus image to be recognized;
inputting the fundus feature map into the attention mechanism model, and obtaining an attention-weighted feature map; and
obtaining a target disease label of the fundus image to be recognized by invoking the classification model to classify the attention-weighted feature map.

17. The electronic device according to claim 16, wherein the attention mechanism model comprises a perception layer, “inputting the fundus feature map into the attention mechanism model, and obtaining an attention-weighted feature map” comprises:
performing a pooling processing on the fundus feature map and obtaining pooling values;
inputting each of the pooling values into the perception layer, and obtaining a target weight vector of the fundus feature map based on a weight of each neuron in the perception layer for each pooling value; and
generating the attention-weighted feature map based on the fundus feature map and the target weight vector.

18. The electronic device according to claim 17, wherein “performing a pooling processing on the fundus feature map and obtaining pooling values; inputting each of the pooling values into the perception layer, and obtaining a target weight vector of the fundus feature map based on a weight of each neuron in the perception layer for each pooling value” comprises:
performing a global average pooling on the fundus feature map and obtaining global average pooling values;
inputting each of the global average pooling values into the perception layer, and obtaining the target weight vector based on a weight of each neuron in the perception layer for each global average pooling value; and
generating the attention-weighted feature map based on the fundus feature map and the target weight vector.

19. The electronic device according to claim 17, wherein the perception layer comprises a first perception layer and a second perception layer, the target weight vector comprises a first target weight vector and a second target weight vector, and “performing a pooling processing on the fundus feature map and obtaining pooling values; inputting each of the pooling values into the perception layer, and obtaining a target weight vector of the fundus feature map based on a weight of each neuron in the perception layer for each pooling value” comprises:
performing a global average pooling processing on the fundus feature map and obtaining global average values;
inputting each of the global average values into the first perception layer, and obtaining a first target weight vector based on a weight of each neuron in the first perception layer for each global average value; and
performing a global maximum pooling processing on the fundus feature map and obtaining global maximum values; and
inputting each global maximum value into the second perception layer, and obtaining a second target weight vector based on a weight of each neuron in the second perception layer for each global maximum value.

20. The electronic device according to claim 19, wherein the at least one processor generates the attention-weighted feature map based on the fundus feature map and the target weight vector by:
determining a first attention feature map based on the fundus feature map and the first target weight vector;
determining a second attention feature map based on the fundus feature map and the second target weight vector; and
generating the attention-weighted feature map based on the first attention feature map and the second attention feature map.
